(19) United States
(12) Patent Application Publication
(10) Pub. No.: US 2024/0165047 A1
(43) Pub. Date: May 23, 2024

(54) Use Of Honokiol In Preparation Of Drug For Treating Meningioma

(71) Applicant: Chengdu Jinrui Foundation Biotech Co., Ltd., Chengdu, Sichuan (CN)

(72) Inventors: Wenbin Li, Chengdu, Sichuan (CN); Ce Wang, Chengdu, Sichuan (CN); Xintian Lai, Chengdu, Sichuan (CN); Xiaobo Wang, Chengdu, Sichuan (CN)

(73) Assignee: Chengdu Jinrui Foundation Biotech Co., Ltd., Chengdu, Sichuan (CN)

(21) Appl. No.: 18/282,410

(22) PCT Filed: Aug. 11, 2022
(86) PCT No.: PCT/CN2022/111765

§ 371 Date: Sep. 15, 2023

(30) Foreign Application Priority Data
Aug. 12, 2021 (CN) 202110924249.1

(51) Int. Cl. A61K 31/05; A61K 9/00; A61K 9/127; A61P 35/00

(52) U.S. Cl. CPC A61K 31/05; A61K 9/0019; A61K 9/127; A61P 35/00

(57) ABSTRACT

The present invention relates to a medical use of honokiol, and in particular to a use of honokiol in the preparation of a drug for treating a meningioma. Experiments prove that honokiol can effectively inhibit the growth of a meningioma, and eliminate and/or reduce lesions of the meningioma. Moreover, clinical experiments show that the safety and tolerance of honokiol are good.

### TECHNICAL FIELD

The present invention relates to a medical use of honokiol. Specifically, the present invention relates to a use of honokiol in the treatment of meningioma.

### BACKGROUND

The meningioma is the most common primary tumor of the central nervous system, mostly originating from the endothelial cells of the arachnoid, mainly occurring in the granules or villus of the arachnoid. The meningioma accounts for about 36.6% of all primary central nervous system tumors and 53.2% of non-malignant primary central nervous system tumors. Meningiomas are divided into benign meningiomas and malignant meningiomas. The typical clinical symptoms of the meningioma are increased intracranial pressure, focal nervous system (including cranial nerve) defects, and generalized and partial epileptic seizures caused by focal mass effect, the most common of which are headache, epileptic seizures, visual symptoms, limb weakness, and altered mental status.

The incidence of the meningioma has a tendency to increase year by year, from 4.52/100,000 during 1998-2002 to 8.3/100,000 during 2010-2014. The onset of the meningioma is more common in elderly patients, mostly 70 to 80 years old.

Ionizing radiation is a risk factor for the development of the meningioma. In Hiroshima and Nagasaki, 88 meningiomas were observed among 80,160 atomic bomb survivors. Even very small doses of radiation to the head (eg, those used in dental radiography) may increase the risk of the development of the meningioma. However, there is no clear dose-response relationship between ionizing radiation and the incidence of the meningioma. Epidemiological studies have shown that a history of head trauma, smoking, and cell phone use has not been shown to be associated with an increased risk of the meningioma.

Currently, the standard protocol for treating meningioma generally includes surgery and radiotherapy. Although progress has been made in surgery and radiotherapy, most patients can be cured, but the recurrence rate of the malignant meningioma is as high as 50% to 80%, and the median survival time of some patients is less than 2 years. The disease is very difficult to treat. So far, chemotherapy, hormone therapy and immunotherapy have had very limited efficacy in patients with the malignant meningiomas; due to the particularity of brain tumors, surgical treatment for recurrent patients is also very limited; and surgery, radiotherapy and chemotherapy make patients poorly tolerated. With the aging of the population, the incidence of the meningioma increases. Effective therapies for meningiomas with good safety and tolerability are becoming more clinically important and urgently needed to be developed.

### SUMMARY

Regarding the deficiencies in the treatment of meningiomas in the prior art, the purpose of present invention is to develop a method that can effectively treat meningiomas, and the method has good safety and tolerance.

Honokiol, the English name is Honokiol, the chemical name is 3′,5-di-2-propenyl-1,1′-biphenyl-2,4′-diphenol, and the structural formula is as shown in formula (1):

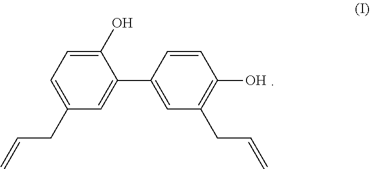 [SMILES: C=CCc1ccc(O)c(-c2ccc(O)c(CC=C)c2)c1]

Honokiol is a small molecular compound with a wide range of biological activities extracted and isolated from the bark of Magnolia officinalis Rehd.etWils. The main biological activities of honokiol include anti-inflammation, anti-microbial, anti-ulcer, anti-oxidation, anti-anxiety, anti-depression, anti-thrombotic, anti-aging and cholesterol-lowering, etc.

Regarding the extensive medicinal value of honokiol, the inventor further studied the new use of honokiol. In clinical research, the inventor found that honokiol can effectively inhibit the growth of the meningioma, and eliminate and/or reduce lesions of the meningioma.

Therefore, one of the purposes of the present invention is to provide a use of honokiol in the preparation of a drug for treating meningioma.

According to the use for treating the meningioma of the present invention, the honokiol is prepared as a honokiol liposome, preferably a honokiol liposome for injection.

According to the use for treating the meningioma of the present invention, the honokiol inhibits the cell proliferation of the meningioma.

Another object of the present invention is to provide a honokiol liposome for treating meningioma.

According to the honokiol liposome of the present invention, the honokiol liposome is the honokiol liposome for injection.

According to the honokiol liposome of the present invention, the honokiol liposome includes following dosage forms: freeze-dried powder preparations, including injection freeze-dried powder preparations and oral freeze-dried powder preparations; tablets, including immediate-release tablets and sustained-release tablets; capsules, including hard capsules, soft capsules, sustained-release capsules and enteric-coated capsules; or transdermal preparations, etc.

According to the honokiol liposome of the present invention, the honokiol liposome can be administered through following routes: intravenous injection, intramuscular injection, subcutaneous injection, oral administration, eye administration, pulmonary administration, transdermal administration and nasal administration, etc.

### Beneficial Effect

It has been found in clinical trials that honokiol can effectively treat the meningioma, effectively inhibit the growth of the meningioma, and eliminate and/or reduce lesions of the meningioma. Moreover, clinical trials have shown that honokiol has good safety and tolerance.

### DESCRIPTION OF DRAWINGS

FIGS. 1A to 1C are the cranial magnetic resonance images of the meningioma patient before treatment with honokiol and after one cycle and three cycles of treatment with honokiol in Example 1, respectively.

FIGS. 2A to 2C are the cranial magnetic resonance images of the meningioma patient before treatment with honokiol and after one cycle and three cycles of treatment with honokiol in Example 2, respectively.

FIGS. 3A to 3C are the cranial magnetic resonance images of the meningioma patient before treatment with honokiol and after one cycle and three cycles of treatment with honokiol in Example 3, respectively.

FIGS. 4A to 4C are the cranial magnetic resonance images of the meningioma patient before treatment with honokiol and after one cycle and three cycles of treatment with honokiol in Example 4, respectively.

### DETAILED DESCRIPTION

Examples are provided below to further illustrate the use of honokiol in the preparation of a drug for treating meningioma.

The abbreviations of the technical terms involved in the examples are listed in Table 1 below.

TABLE 1

| | |
|---|---|
| ALT | alanine aminotransferase |
| APTT | activated partial thromboplastin time |
| AST | aspertate aminotransferase |
| CCr | creatinine clearance rate |
| CD34 | cluster of differentiation 34 |
| CD99 | cluster of differentiation 99 |
| CgA | chromogranin A |
| CK | cytokeratin |
| CPA | cerebellopontine angle |
| CTCAE | Common Terminology Criteria for |
| | Adverse Events |
| DLT | dose-limiting toxicity |
| ECOG | Eastern Cooperative Oncology Group |
| EMA | epithelial membrane antigen |
| Ki67 | cell proliferation marker |
| KPS | Karnofsky physical score |
| LVEF | left ventricular ejection fraction |
| MR | magnetic resonance |
| PR | progesterone receptor |
| PT | prothrombin time |
| RANO | response assessment in |
| | neuro-oncology |
| RECIST1.1 | Response Evaluation Criteria in Solid |
| | Tumors Version 1.1 |
| S100 | nervous tissue-origin marker |
| SAE | serious adverse event |
| SD | stable disease |
| SSTR | somatostatin receptor |
| Syn | synaptophysin |
| TT | thrombin time |
| ULN | upper limit of normal |

The test material used in the example is as follows:

Honokiol liposome for injection is derived from Chengdu JinRui Foundation Biotechnology Co., Ltd., or can be prepared according to the following methods: 50 mg of honokiol, 500 mg of soybean lecithin, 200 mg of cholesterol, and 200 mg of peony phosphatidylethanolamine were dissolved in 50 mL of absolute ethanol for complete dissolution, the obtained solution was injected into 300 mL of purified water and stirred, ethanol was then removed by rotary evaporation, and 800 mg of sucrose as a freeze-dried excipient was added, lyophilized.

### Example 1

Description of the Subject:
male, 20 years old;
on Jan. 20, 2018, the subject's cranial MR showed the space-occupying of CPA on the right side and the jugular foramen region; on Nov. 27, 2018, the subject underwent the resection of a deep skull bottom lesion, endothelial meningoma, and local lesion being hemangioma-type meningoma, accompanied by bleeding, showing the non-typical meningioma, infiltrating bone tissue, comprehensive to malignant meningoma; immunohistochemistry: EMA (positive), PR (scattered in a little positive), SSTR (focal positive), CD34 (blood vessel positive), CD99 (+), S100 (−), Syn (−), CgA (−), CK (occasionally positive), Ki67 (about 8-20%);
in February 2019, the subject was treated for intracranial infection;
in June 2019, the subject received whole brain radiotherapy;
on Mar. 5, 2021, the subject re-examined the cranial MR and the tumor recurred; in order to further treat, at the outpatient of Beijing Tiantan Hospital, Capital Medical University, the subject was included in the comprehensive therapy ward of neural tumor for “malignant tumor maintenance treatment”;
on May 13, 2021, the subject entered the group “Phase I clinical trial of the clinical safety and tolerance of the injecting honokiol liposome (HK) for the treatments of the patients with advanced malignant solid tumors”, the selected standard is as follows:
1) 18 years old≤age≤80 years old, gender is not limited;
2) for the patients with advanced malignant solid tumors diagnosed by histology or cytology, the previous standard treatment plans failed, they cannot tolerate or refuse existing therapy, preferably tumor species such as lung cancer, liver cancer, glioma, ovarian cancer, and colon cancer;
3) according to the RECIST 1.1 standard (or RANO standard), there are evaluated tumor lesions;
4) except for glioma, patients with brain metastases selected for other tumor species must meet the following conditions: no clinical symptoms related to brain metastases, no need for systemic corticosteroids or anticonvulsant drug treatment; patients with brain metastases who have been treated must be no progress by re-examination after 28 days at the end of treatment; no risk of intracerebral hemorrhage;
5) those without severe hematopoietic dysfunction (absolute value of neutrophils≥1.5×10⁹/L, platelets≥80×10⁹/L, hemoglobin≥100 g/L);
6) those without serious organic disease of heart, lung, liver and kidney (LVEF (left ventricular ejection fraction) ≥50%; total bilirubin≤1.5×ULN; alanine aminotransferase (ALT)≤2.5×ULN (≤5×ULN if there is liver metastasis); aspartate aminotransferase (AST)≤2.5×ULN (≤5×ULN if there is liver metastasis); serum creatinine≤1.5×ULN or CCr>40 mL/min);
7) those without severe coagulation abnormalities (PT≤1.5×ULN, APTT≤1.5×ULN, TT≤1.5×ULN);
8) at least 4 weeks have passed since the patients received the last anti-tumor treatment (chemotherapy, radiotherapy, biological therapy, hormone therapy and targeted therapy) before entering the group;
9) expected survival time ≥12 weeks;
10) for patients with glioma, KPS score >50 points;
11) for patients with other solid tumors, the ECOG score≤1;
12) those who agree to participate in this study and sign the informed consent form.

Three cycles of medication were given to the subject: a cycle of 28 days, in which 5 days of continuous administration per week, 2 days of drug withdrawal, a period of 3 weeks, and 1 week of rest; honokiol liposome for injection was administered, and the dosage is 420 mg/day honokiol; the method of administration is administration via peripherally inserted central venous catheters, and the infusion time is 2 hours.

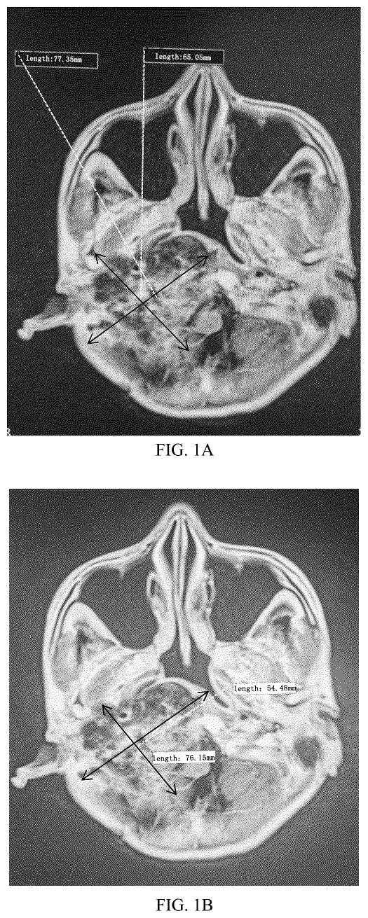

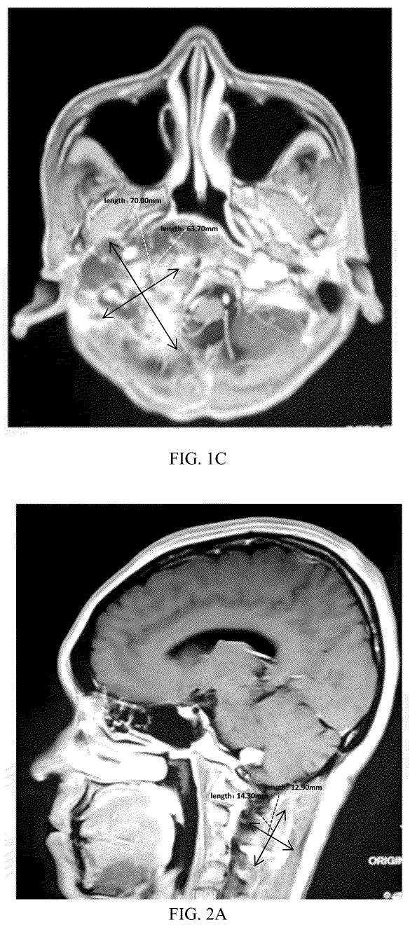

After completing the one cycle of medication, cranial MR re-examination was conducted. FIG. 1A and FIG. 1B show the cranial MR comparison images before and after the treatment. According to the RANO standard, the target lesion was reduced by 17.46% compared with that before treatment, and the product of the two perpendicular diameters of the target lesion was reduced from 77.3 mm×65 mm before treatment to 76.1 mm×54.5 mm. After completing the three cycles of medication, the cranial MR re-examination was conducted, and the cranial MR image is shown in FIG. 1C. According to the RANO standard, it showed that the target lesion was reduced by 11.13% compared with that before treatment, and the product of the two perpendicular diameters of the target lesion was reduced to 70.1 mm×63.7 mm. The curative effect was evaluated as stable disease (SD), with improvement in clinical performance and no adverse reaction.

As of May 31, 2021, according to the inclusion standard of the above “Phase I clinical trial of the clinical safety and tolerance of the injecting honokiol liposome (HK) for the treatments of the patients with advanced malignant solid tumors” in Example 1, 37 subjects were included in the safety analysis set, and were administered different doses of honokiol liposomes for injection. The doses were 20 mg/day for 4 cases, 40 mg/day for 3 cases, 80 mg/day for 4 cases, 140 mg/day for 3 cases, 210 mg/day for 5 cases, 300 mg/day for 6 cases, and 420 mg/day for 12 cases. The administration method is administration via peripherally inserted central venous catheters, and the infusion time is 2 hours. Every 4 weeks is one administration cycle, 3 weeks per administration cycle (the first week to the third week of this cycle), 5 days of continuous administration per week, once a day, 2 days of drug withdrawal, and 1 week of rest.

After administration of honokiol liposome for injection, the main adverse reactions of the subjects were neutropenia, constipation, anemia, increased ALT, decreased fibrinogen, headache, dizziness, and hypokalemia. Most of these adverse reactions were mild. There was 1 case of DLT (CTCAE grade 3 increased ALT), and 4 cases of SAE were observed, all of which were remission, and there were no suspicious unexpected serious adverse reactions. Adverse events, adverse reactions, incidence of SAE, subjects' vital signs, physical examination, laboratory examination, 12-lead electrocardiogram, echocardiogram and other clinically significant changes were not evidenced by an increasing trend with increasing dose.

Clinical trials have shown that honokiol liposomes for injection can not only effectively treat the meningioma, but also have good safety and tolerance.

### Example 2

Description of the Subject:
female, 39 years old;

One year before admission, the patient had pain and discomfort in the neck and pillow area without obvious incentives, which aggravated after exercise, without limb movement disorders, and without obvious upper limb radiating pain, which aggravated at night. For further treatment, she went to the Second Hospital of Dalian Medical University, and a cranial MRI showed a space occupying of the medulla. During the perioperative treatment, she went to the Second Hospital of Dalian Medical University. On Jan. 10, 2020, lower brainstem tumor resection under general anesthesia was performed in this hospital. During the operation, the tumor size was about 1.5*1.5 cm, accompanied by tumor hemorrhage, rotten fish-like, rich blood supply, and the tumor was completely resected under the microscope. Postoperative pathology: non-typical meningioma (WHO-II), KI-67 (25%). Hoarseness occurred postoperatively.

Before admission, she felt dizzy, weak, and occasionally coughed. She went to Tiantan Hospital, and a cranial MRI was performed 1 week before admission, showing that the medullary lesions had progressed. Two cycles of self-provided PD-1 immunotherapy were given, and the process went smoothly. Now for further treatment, the outpatient is hospitalized with “meningioma”.

On Oct. 14, 2021, as described in Example 1, “Phase I clinical trial of the clinical safety and tolerance of the injecting honokiol liposome (HK) for the treatments of the patients with advanced malignant solid tumors”, she was selected into the standard group for administration.

Three cycles of medication were given to the subject: a cycle of 28 days, in which 5 days of continuous administration per week, 2 days of drug withdrawal, a period of 3 weeks, and 1 week of rest; honokiol liposome for injection was administered, and the dosage is 420 mg/day honokiol; the method of administration is administration via peripherally inserted central venous catheters, and the infusion time is 2 hours.

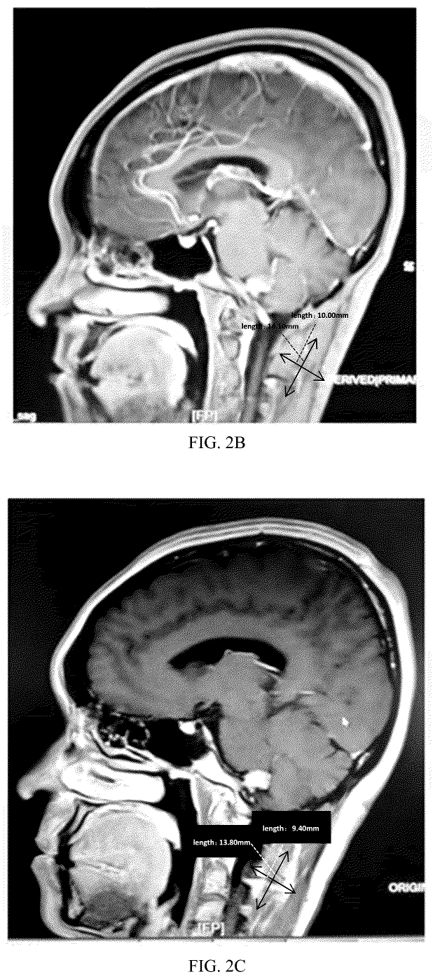

After completing the one cycle of medication, the cranial MR re-examination was conducted. FIG. 2A and FIG. 2B show the cranial MR comparison images before and after the treatment. According to the RANO standard, the target lesion was reduced by 22.97% compared with that before treatment, and the product of the two perpendicular diameters of the target lesion was reduced from 14.3 mm×12.9 mm before treatment to 14.1 mm×10 mm. After completing the three cycles of medication, the cranial MR re-examination was conducted, and the cranial MR image is shown in FIG. 2C. According to the RANO standard, it showed that the target lesion was reduced by 29.13% compared with that before treatment, and the product of the two perpendicular diameters of the target lesion was reduced to 13.8 mm×9.4 mm. The curative effect was evaluated as stable disease (SD), with clinical manifestations of stability and tolerance to the drug.

### Example 3

Description of the Subject:
male, 53 years old;

Five years before admission, the patient had no obvious cause of “twitching of the limbs accompanied by foaming at the mouth, and rolling his eyes for 4 hours”, no memory loss, no headache, dizziness, no nausea, vomiting, no limb movement disorder, no hearing and vision loss. The cranial MRI showed intracranial space occupying, and he went to the First People's Hospital of Jining to complete relevant examinations. On Feb. 21, 2016, a right frontal tumor resection was performed, and the postoperative pathology: meningioma. Gamma knife treatment was performed once in Shandong Armed Police General Hospital after operation. Cranioplasty was performed in October 2017.

On Feb. 13, 2019, an epileptic seizure occurred, and the tumor recurrence was considered by re-examination. On Feb. 18, 2019, the embolization of pelvic artery of the meningioma was performed. On Feb. 21, 2019, the original incision approach tumor resection was performed. Postoperative pathology: meningioma (WHO I), postoperative whole brain radiotherapy was given. In April 2021, the tumor recurrence was considered by re-examination and no treatment was given. In May 2021, an epileptic seizure occurred and Debakin was given for treatment. In June 2021, he had a major epileptic seizure, which improved after symptomatic treatment, and the cranial MRI showed larger tumor than before.

On Jul. 21, 2021, resection of the tumor next to the right frontal lobe was performed again. Postoperative pathology: anaplastic meningioma (WHO Ill), Ki-67 (50%). On Aug. 17, 2021, one cycle of tislelizumab was administered. For further treatment, the outpatient is hospitalized with “meningioma”.

On Oct. 21, 2021, as described in Example 1, “Phase I clinical trial of the clinical safety and tolerance of the injecting honokiol liposome (HK) for the treatments of the patients with advanced malignant solid tumors”, he was selected into the standard group for administration.

Three cycles of medication were given to the subject: a cycle of 28 days, in which 5 days of continuous administration per week, 2 days of drug withdrawal, a period of 3 weeks, and 1 week of rest; honokiol liposome for injection was administered, and the dosage is 420 mg/day honokiol; the method of administration is administration via peripherally inserted central venous catheters, and the infusion time is 2 hours.

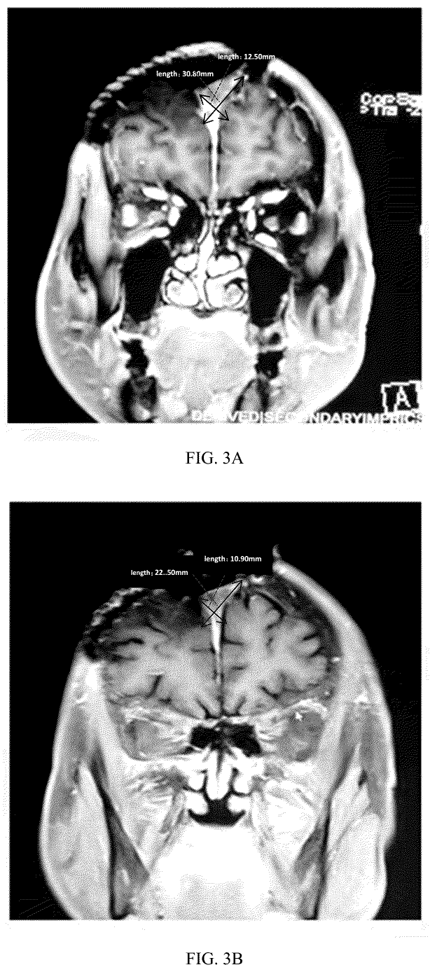

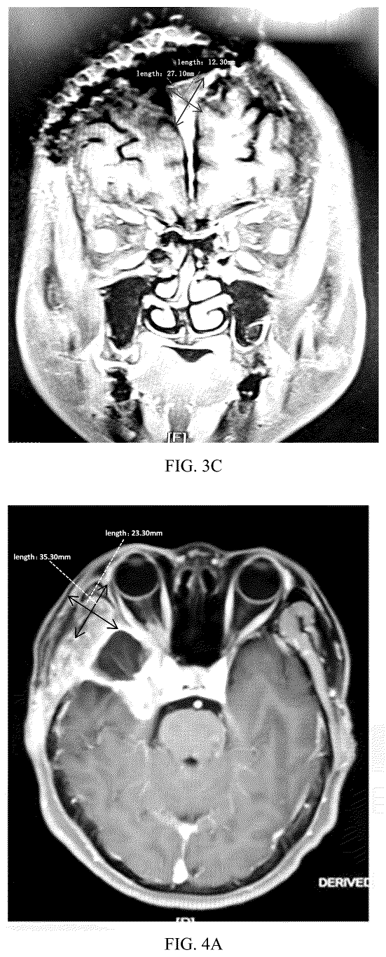

After completing the one cycle of medication, the cranial MR re-examination was conducted. FIG. 3A and FIG. 3B show the cranial MR comparison images before and after the treatment. According to the RANO standard, the target lesion was reduced by 35.30% compared with that before treatment, and the product of the two perpendicular diameters of the target lesion was reduced from 12.5 mm×30.8 mm before treatment to 10.9 mm×22.5 mm. After completing the three cycles of medication, the cranial MR re-examination was conducted, and the cranial MR image is shown in FIG. 3C. According to the RANO standard, it showed that the target lesion was reduced by 13.42% compared with that before treatment, and the product of the two perpendicular diameters of the target lesion was reduced to 12.3 mm×27.1 mm. The curative effect was evaluated as stable disease (SD), with improvement in clinical manifestations and tolerance to the drug.

### Example 4

Description of the Subject:
female, 53 years old;

The patient went to the local hospital in 2012 due to epileptic seizures, and the examination revealed an intracranial space occupying. Right frontotemporal craniotomy and space occupying resection was performed, postoperative pathology: low meningioma of right middle cranial fossa (WHO I), regular re-examination every year after operation.

In 2016 and 2018, Gamma Knife treatments were performed due to recurrence. One year ago, the vision on the right side gradually declined, and then intermittent headaches, no limb convulsions, no incontinence, no nausea and vomiting, no limb movement disorders. She went to the local hospital 3 months before admission, the cranial MRI was performed, and it was found that the postoperative changes of the right frontotemporal craniotomy, space occupying in the right sphenoid ridge, middle cranial fossa, infratemporal fossa and right orbit: recurrence of the meningioma with multiple enhancements at the right orbital apex, anterior cranial fossa, and right tentorial marginal; multiple cerebral ischemic white matter lesions. Pathology: anaplastic meningioma (WHO III), Ki-67 (about 40-60%). For further treatment, the outpatient is hospitalized with “meningioma”.

On Oct. 28, 2021, as described in Example 1, “Phase I clinical trial of the clinical safety and tolerance of the injecting honokiol liposome (HK) for the treatments of the patients with advanced malignant solid tumors”, she was selected into the standard group for administration.

Three cycles of medication were given to the subject: a cycle of 28 days, in which 5 days of continuous administration per week, 2 days of drug withdrawal, a period of 3 weeks, and 1 week of rest; honokiol liposome for injection was administered, and the dosage is 420 mg/day honokiol; the method of administration is administration via peripherally inserted central venous catheters, and the infusion time is 2 hours.

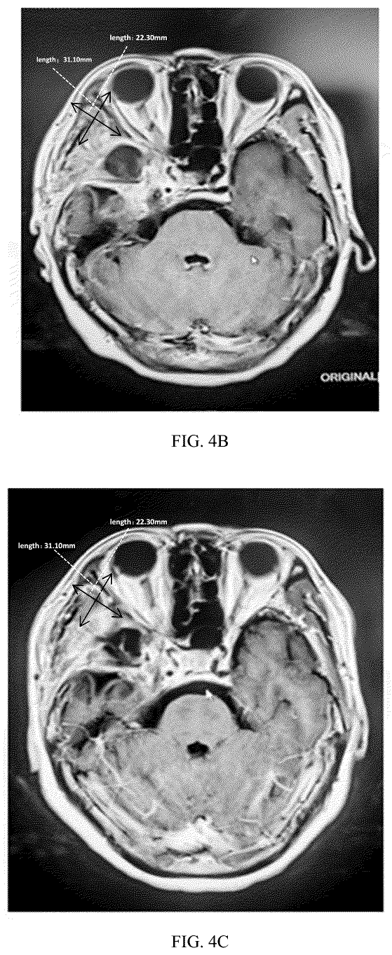

After completing the one cycle of medication, the cranial MR re-examination was conducted. FIG. 4A and FIG. 4B show the cranial MR comparison images before and after the treatment. According to the RANO standard, the target lesion was reduced by 15.68% compared with that before treatment, and the product of the two perpendicular diameters of the target lesion was reduced from 23.3 mm×35.3 mm before treatment to 22.3 mm×31.1 mm. After completing the three cycles of medication, the cranial MR re-examination was conducted, and the cranial MR image is shown in FIG. 4C. According to the RANO standard, it showed that the target lesion was reduced by 15.68% compared with that before treatment, and the product of the two perpendicular diameters of the target lesion was reduced to 22.3 mm×31.1 mm. The curative effect was evaluated as stable disease (SD), with improvement in clinical manifestations and tolerance to the drug.

1. A method for treating meningioma, comprising administering honokiol to a subject in need thereof.

2. The method according to claim 1, wherein the honokiol is prepared as a honokiol liposome.

3. The method according to claim 2, wherein the honokiol liposome is a honokiol liposome for injection.

4. The method according to claim 1, wherein the honokiol inhibits a cell proliferation of the meningioma.

5. A honokiol liposome for treating meningioma.

6. The honokiol liposome according to claim 5, wherein the honokiol liposome is a honokiol liposome for injection.

7. The honokiol liposome according to claim 5, wherein the honokiol liposome comprises following dosage forms: freeze-dried powder preparations, including injection freeze-dried powder preparations and oral freeze-dried powder preparations; tablets, including immediate-release tablets and sustained-release tablets; capsules, including hard capsules, soft capsules, sustained-release capsules and enteric-coated capsules; or transdermal preparations.

8. The honokiol liposome according to claim 5, wherein the honokiol liposome is administered through following routes: intravenous injection, intramuscular injection, subcutaneous injection, oral administration, eye administration, pulmonary administration, transdermal administration and nasal administration.
